(19) United States
(12) Patent Application Publication
(10) Pub. No.: US 2010/0179626 A1
(43) Pub. Date: Jul. 15, 2010

(54) SYSTEM AND METHOD FOR IMPLANTING A PADDLE LEAD

(75) Inventors: Craig S. Pilarski, Ham Lake, MN (US); Thomas I. Miller, Plymouth, MN (US); Christopher J. Poletto, North Oaks, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/683,726

(22) Filed: Jan. 7, 2010

Related U.S. Application Data
(60) Provisional application No. 61/143,428, filed on Jan. 9, 2009.

(51) Int. Cl. A61N 1/05

(52) U.S. Cl. 607/116

(57) ABSTRACT

A method includes
(i) inserting a paddle portion of a lead into a longitudinal slit of a tool having a lumen in communication with the slit;
(ii) axially rotating the tool relative to the paddle portion of the lead to cause the paddle portion of the lead to enter the lumen;
(iii) axially aligning the lumen of the tool with a lumen of an introducer; and
(iv) advancing the paddle portion of the lead through the lumen of the tool and into the lumen of the introducer.

### RELATED APPLICATION

This application claims priority to U.S. Provisional Application No. 61/143,428, filed Jan. 9, 2009. U.S. Provisional Application No. 61/143,428 is hereby incorporated herein by reference in its entirety.

### FIELD

The present disclosure relates to implantable medical devices; more particularly to surgical or paddle medical leads and methods of implantation thereof.

### BACKGROUND

Implantable medical systems capable of generating therapeutic electrical signals are used to treat a variety of disorders. Such systems employ implantable electrical signal generators, often referred to pulse generators, to generate the therapeutic signal. A medical lead is used to deliver the signal from the generator to a target location of a patient. A variety of types of medical leads exist, with each type having advantages and disadvantages based on the purpose for which it is employed.

One type of implantable medical lead is commonly referred to as a percutaneous lead. Such leads are elongate leads typically having a circular radial cross sectional shape. Percutaneous leads often contain ring shaped electrodes at or near their distal ends that allow an electrical signal to be applied to tissue via the electrodes in 360 degrees. The outer diameters of percutaneous leads tend to be substantially uniform along their length, which lends to an ability to be delivered to a tissue location via a needle or introducer. Following positioning of the distal end of the lead in a target tissue through a needle or introducer, the needle or introducer can readily be withdrawn over the lead. Such an implantation method can be relatively non-invasive and may require only a small or no incision.

Another type of implantable medical lead is commonly referred to as a surgical or paddle lead. Such leads often include a paddle-shaped distal portion having electrodes exposed through one face of the paddle portion. Such paddle or surgical leads deliver therapeutic electrical signals to tissue in a directional manner. Because of their paddle-shaped distal portions, surgical leads do not lend themselves to delivery via a needle or introducer. Rather, large incisions are typically required to properly position distal portions of paddle leads. Such large incisions can lead to complications such as infection and patient discomfort.

### BRIEF SUMMARY

The present disclosure, among other things, describes paddle leads, systems and methods for delivering paddle leads through a needle or introducer in a manner similar to percutaneous leads.

In various embodiments, a system or kit is described. The system includes a tool having a body member defining a proximal end, a distal end, a lumen extending from the proximal end to the distal end, and a longitudinal slit in communication with the lumen and extending from the proximal end towards the distal end. The system further includes a lead having (i) a proximal end including a contact for operably coupling the lead to an active electrical medical device, and (ii) a paddle-shaped distal end portion including an electrode electrically coupled to the contact. The system also includes an introducer having a body member defining a proximal end, a distal end, and a lumen extending from the proximal end to the distal end. The tool and lead are configured such that axial rotation of the tool relative to the lead when the paddle portion of the lead is received by the slit causes the paddle portion of be fully received by the lumen. The tool, the lead, and the introducer are configured such that the paddle portion of the lead is advanceable distally through the lumen of the tool and into the lumen of the introducer when the lumens of the tool and the introducer are axially aligned.

In numerous embodiments, a method is described. The method includes (i) inserting a paddle portion of a lead into a longitudinal slit of a tool having a lumen in communication with the slit; (ii) axially rotating the tool relative to the paddle portion of the lead to cause the paddle portion of the lead to enter the lumen; (iii) axially aligning the lumen of the tool with a lumen of an introducer; and (iv) advancing the paddle portion of the lead through the lumen of the tool and into the lumen of the introducer.

In various embodiments, a method for applying an electrical signal to an occipital nerve of a patient includes positioning a distal end of an introducer in proximity to an occipital nerve of a patient. The introducer has a proximal end, the distal end, and a lumen extending from the proximal end to the distal end. The method further includes (i) inserting a paddle portion of a lead into a longitudinal slit of a tool having a lumen in communication with the slit; (ii) axially rotating the tool relative to the paddle portion of the lead to cause the lumen of the tool to fully receive the paddle portion of the lead; (iii) axially aligning the lumen of the tool with a lumen of the introducer; (iv) advancing the paddle portion of the lead through the lumen of the tool and into the lumen of the introducer; (v) distally advancing the paddle portion of the lead through the lumen of the introducer until the paddle portion of the lead is in proximity to the distal end of the introducer and the occipital nerve; (vi) withdrawing the introducer from the patient over the lead; and (vii) operably coupling the lead to an electrical signal generator such that a signal generated by the signal generator is deliverable to the occipital nerve via the paddle portion of the lead.

In some embodiments, a method is described where a distal paddle-shaped portion of a lead is bent and inserted into a lumen of an introducer.

In some embodiments, a method is described where a paddle portion of a lead is inserted into a longitudinal slit of an introducer. The longitudinal slit is in communication with a lumen of the introducer. The method includes axially rotating the introducer to cause the paddle portion of the lead to enter a lumen of the introducer.

Advantages of the various systems, devices and methods described herein will be readily understood from the following detailed description when read in conjunction with the accompanying drawings.

### BRIEF DESCRIPTION OF THE DRAWINGS

FIG. 1 is a schematic side view of a representative implantable system for delivering therapeutic electrical signals.

FIG. 2A is a schematic side view of a medical lead having a paddle-shaped distal portion.

FIG. 2B is a schematic cross sectional view of an embodiment of the lead shown in FIG. 2A taken through line 2B-2B.

FIG. 2C is a schematic cross sectional view of an embodiment of the lead shown in FIG. 2A taken through line 2C-2C.

FIG. 3A is a schematic side view of a representative introducer.

FIG. 3B is a schematic cross sectional view of an embodiment of the introducer shown in FIG. 3A taken through line 3B-3B.

FIG. 4 is a schematic cross sectional view of a representative bent paddle portion of a lead fully received in a lumen of a representative introducer.

FIGS. 5A-D are schematic cross sectional views showing representative selected steps of implanting a lead via an introducer.

FIG. 6 is a schematic view showing representative distal portions of surgical or paddle leads implanted in a patient to apply electrical signals to left and right occipital nerves.

FIGS. 7-8 are schematic side views of embodiments of representative introducers.

FIGS. 9A-C are schematic cross sectional views showing a representative paddle portion of a lead and a representative introducer.

FIG. 10 is a schematic view showing a representative distal portion of a bifurcated surgical or paddle lead implanted in a patient to apply electrical signals to left and right occipital nerves.

FIG. 11 is a schematic side view of a representative bifurcated surgical or paddle lead.

FIG. 12 is a schematic perspective view of a tool for facilitating implantation of a surgical or paddle lead via an introducer.

FIGS. 13A-C are schematic cross sectional views showing a representative paddle portion of a lead and a representative tool.

FIG. 14 is a schematic side view of a representative tool and introducer.

FIGS. 15A-B are schematic side views of a representative tool and introducer having a proximal hub, where the tool is in proximity to (15A) or received by (15B) the hub.

FIG. 15C is a schematic cross sectional view of an embodiment of a tool and introducer shown in FIG. 15B taken through line 15A-15A.

FIG. 15D is a schematic view of a longitudinal section of an embodiment of a tool and introducer shown in FIG. 15B, showing the tool and introducer proximal to line 15D-15D.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

The present disclosure describes, inter alia, devices, systems and methods for implanting surgical leads via introducers. Nearly any implantable medical device or system employing surgical or paddle leads may be used in conjunction with the apparatuses, systems and methods described herein. Representative examples of such implantable medical devices include hearing implants, cochlear implants; sensing or monitoring devices; signal generators such as cardiac pacemakers or defibrillators, neurostimulators (such as spinal cord stimulators, brain or deep brain stimulators, peripheral nerve stimulators, vagal nerve stimulators, occipital nerve stimulators, subcutaneous stimulators, etc.), gastric stimulators; or the like. For purposes of occipital nerve stimulation, electrical signal generators such as Medtronic, Inc.'s Restore® or Synergy® series of implantable neurostimulators may be employed.

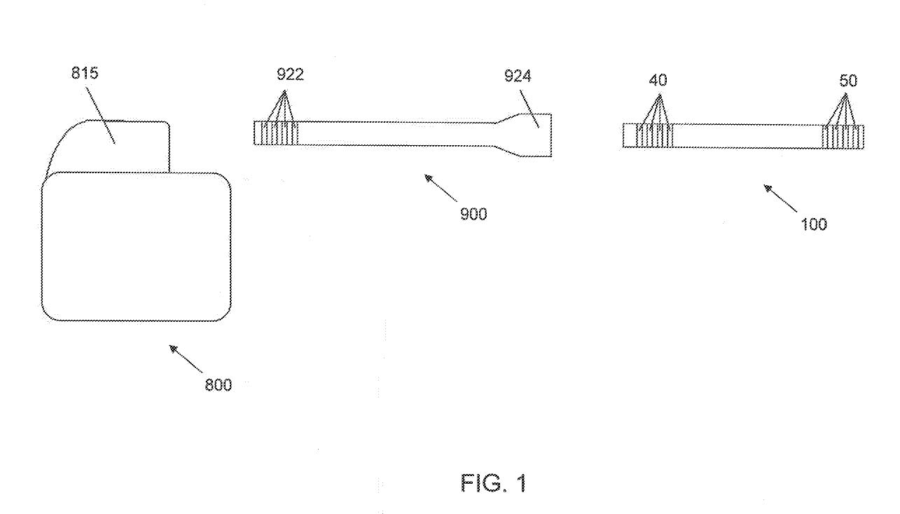

Referring to FIG. 1, a schematic side view of a representative electrical signal generator system is shown. In the depicted system, the electrical signal generator 800 includes a connector header 815 configured to receive a proximal portion of lead extension 900. The proximal portion of lead extension 900 contains a plurality of electrical contacts 922 that are electrically coupled to internal contacts (not shown) at distal connector 924 of lead extension 900. The connector header 815 of the signal generator 800 contains internal contacts (not shown) and is configured to receive the proximal portion of the lead extension 900 such that the internal contacts of the connector header 815 may be electrically coupled to the contacts 922 of the lead extension 900 when the lead extension 900 in inserted into the header 815.

The system depicted in FIG. 1 further includes a lead 100. The depicted lead 100 has a proximal portion that includes a plurality of contacts 40 and a distal portion that includes a plurality of electrodes 50. Each of the electrodes 50 may be electrically coupled to a discrete contact 40. The distal connector 924 of the lead extension 900 is configured to receive the proximal portion of the lead 100 such that the contacts 40 of the lead 100 may be electrically coupled to the internal contacts of the connector 924 of the extension 900. Accordingly, a signal generated by the signal generator 800 may be transmitted to a patient by an electrode 50 of lead 100 when the lead is connected to extension 900 and extension 900 is connected to signal generator 800.

It will be understood that lead 100 may be coupled to signal generator 800 without use of an extension 900. Any number of leads 100 or extensions 900 may be coupled to signal generator 800. Typically, one or two leads 100 or extensions 900 are coupled to signal generator 800. While lead 100 is depicted as having four electrodes 50, it will be understood that lead 100 may include any number of electrodes 50, e.g. one, two, three, four, five, six, seven, eight, sixteen, thirty-two, or sixty-four. Corresponding changes in the number of contacts 40 in lead 100, contacts 922 and internal contacts in connector 924 of lead extension, or internal contacts in header 815 of signal generator 800, may be required or desired.

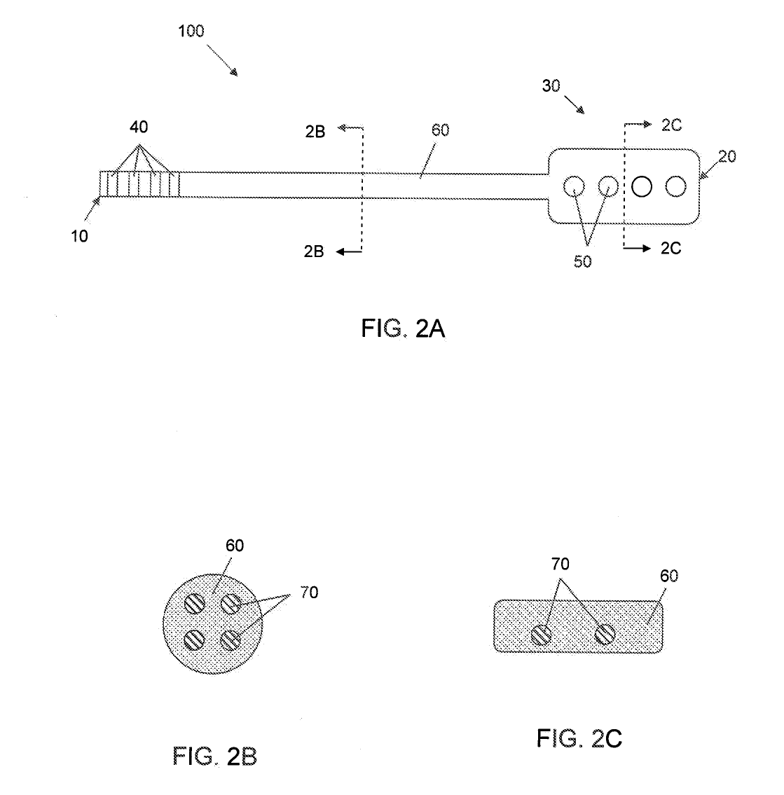

Referring to FIGS. 2A-C, schematics of various views of a lead 100 having a distal paddle-shaped portion 30 are shown. The lead 100 has a proximal end 10 and a distal end 20. Contacts 40 for operably coupling the lead 100 to an active electrical medical device, such as an electrical signal generator, are disposed in proximity to the proximal end 10. Electrodes 50 are disposed in the distal paddle-shaped portion 30. In the depicted embodiment, the electrodes 50 are aligned with the longitudinal midline of the lead 100. The electrodes 50, in the depicted embodiments, are exposed through only one face of the paddle 30 to provide for directional application of an electrical signal to tissue of a patient in which the lead 100 is implanted. However, it will be understood that the electrodes may be exposed through both opposing faces of the paddle 30. Discrete conductors 70 are coupled to discrete contacts 40 and run through lead body 60 to electrodes 50 to which they are electrically coupled. The paddle portion 30 of the lead has a width greater than the outer diameter of a cylindrical portion of the lead 100 proximal the paddle portion 30 (compare, e.g., FIG. 1C to FIG. 1B). The greater width in the paddle portion 30 makes it difficult to deliver such leads 100 to a target location of a patient via an introducer, which typically have a lumen with a diameter capable of slidably receiving leads having uniform outer diameters.

Any suitable paddle or surgical lead may be employed or manufactured and used in accordance with the teachings presented herein. Examples of surgical leads that may be used include Medtronic Inc.'s Resume, SymMix, On-Point, or Specify series of leads. In general, the paddle-shaped portion of a lead used in a system or method described herein should be sufficiently flexible to be inserted into a slit or introducer and advanced through the introducer as discussed in more detail below. In addition, the paddle electrodes may be made from flexible circuits to accommodate flexing of the paddle portion.

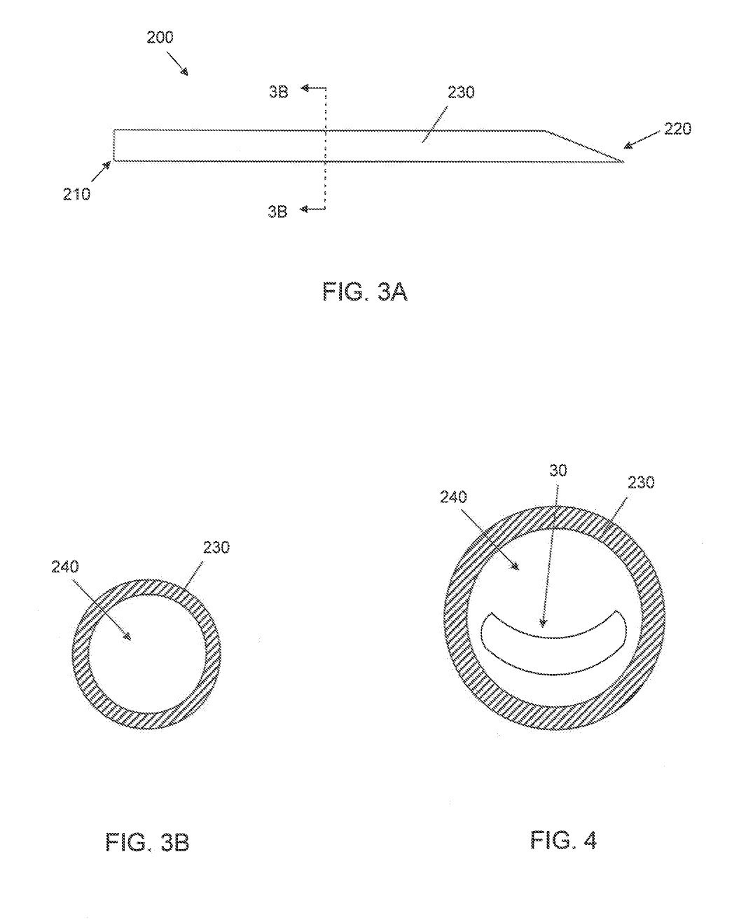

Referring now to FIGS. 3A-B, schematic side (3A) and cross sectional (3B) views of a generic introducer 200 are shown. The introducer 200 includes a body 230 having a proximal end 210, a distal end 220, and defining a lumen 240 extending from the proximal end 210 to the distal end 220. In general, the inner diameters defined by lumens 240 of such introducers 200 are smaller than the widths of paddle portions of surgical leads. Of course it is possible for introducers to be made such that the inner diameter is larger than the width of a paddle portion of a surgical lead; however, the overall radial dimensions of such introducers would be so large that they may not be practical for introducing leads to target tissues of patients.

With reference to FIG. 4, a paddle portion 30 of a lead may be bent, folded, curled or the like so that it may be fully received into a lumen 240 formed by body 230 of an introducer. For purposes of the present disclosure, “bent”, “folded, “curled”, or similar terms are used herein interchangeable and each term is attributed the definition of all such terms unless indicated to the contrary. Once bent and inserted into a lumen 240 of an introducer, the paddle portion 30 of the lead may be advanced distally in the lumen 240, and the introducer may be withdrawn over the lead.

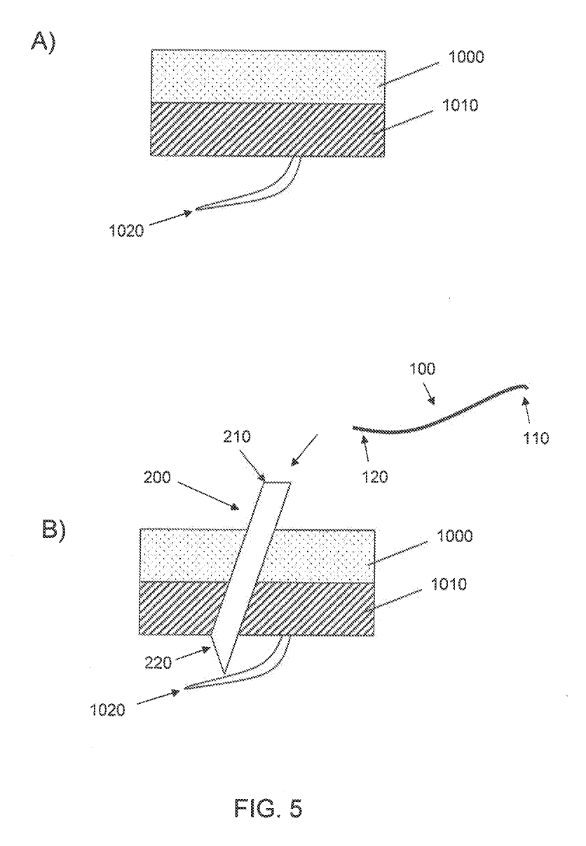

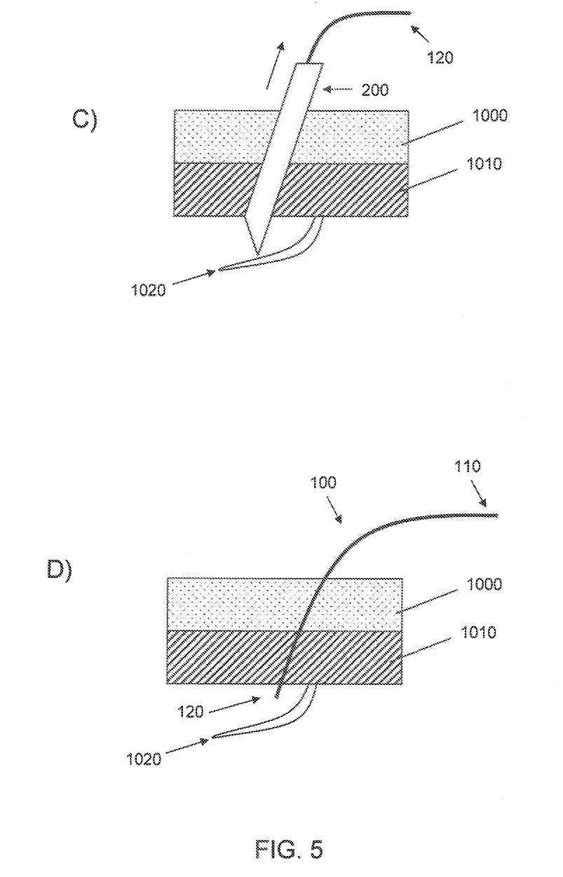

Referring now to FIGS. 5A-D, schematic drawings are presented to show representative steps of lead 100 implant procedure. With reference to FIG. 5A a cross section showing skin 1000, fascia or muscle 1010, and a nerve 1020 of a patient are shown. An introducer 200 is inserted through the skin 1000 and fascia or muscle 1010 such that the distal end 220 is located in proximity to the nerve 1020 and the proximal end 210 of the introducer 200 remains external to the patient (5B). An obturator (not shown) may be inserted into the lumen of the introducer 200 as the introducer 200 is inserted into the patient. A distal end 120 of a lead 100 is inserted into a lumen of the introducer 200 and advanced distally until in proximity with the distal end 220 of the introducer 200 and thus in proximity to the nerve 1020 (5B-C). The introducer 200 may be withdrawn from the patient over the lead 100 leaving the distal end 120 of the lead 100 in proximity to the nerve 1020 (5C-D). It will be understood that FIGS. 5A-D are merely representative for purposes of describing a generic method of implantation. It will be further understood that, in some instances, the targeted nerve may lie between the skin and muscle tissue or within muscle tissue. The angle of insertion of the introducer into the patient may be varied as appropriate or desired depending on the location of the target nerve. In some embodiments, the target nerve is in the muscle layer, while the lead is positioned along the division between the muscle and the skin.

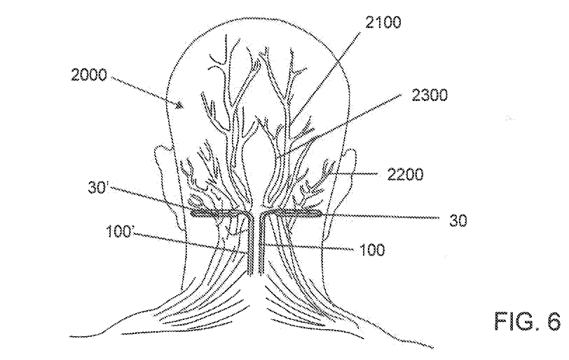

Referring now to FIG. 6, leads 100, 100′ may be implanted in a patient such that distal paddle-shaped portions 30, 30′ having electrodes are positioned to apply an electrical signal to an occipital nerve 2000. As used herein, occipital nerve 2000 includes the greater occipital nerve 2100, the lesser occipital nerve 2200 and the third occipital nerve 2300. The greater and lesser occipital nerves are spinal nerves arising between the second and third cervical vertebrae (not shown). The third occipital nerve arises between the third and fourth cervical vertebrae. The portion of the occipital nerve 2000 to which an electrical signal is to be applied may vary depending on the disease to be treated and associated symptoms or the stimulation parameters to be applied. In various embodiments, the lead distal portions 30, 30′ that contain electrodes are placed to allow bilateral application of electrical signals to the occipital nerve 2000 at a level of about C1 to about C2 or at a level in proximity to the base of the skull. The position of the electrode(s) may vary. In various embodiments, one or more electrodes are placed between about 1 cm and about 8 cm from the midline to effectively provide an electrical signal to the occipital nerve 2000.

To implant the leads 100, 100′so that the distal paddle portions 30, 30′ are positioned to apply signals to an occipital nerve 2000, the paddle-shaped portions 30, 30′ may be implanted via introducers as described above with regard to FIG. 5. The distal paddle portions 30, 30′ may be bent to fit within a lumen of an introducer; e.g. as described above with regard to FIG. 4. While not shown, it will be understood that the introducer may be curved along its length to conform to the head and neck areas in proximity of the occipital nerves to facilitate introduction of the lead. Of course, the introducer may be shaped in any suitable manner to accommodate insertion of a lead to nearly any desirable location of the body.

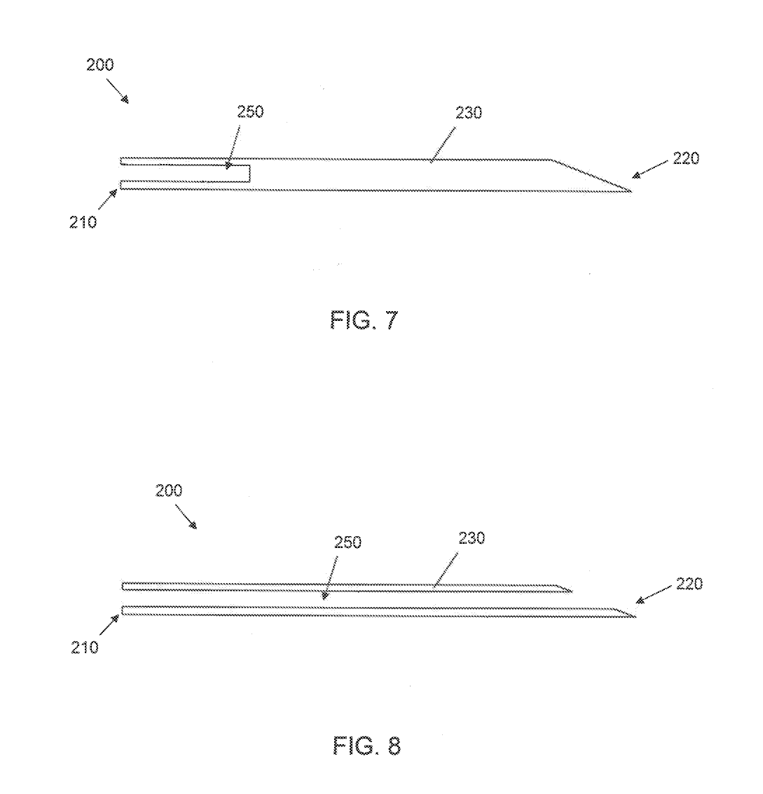

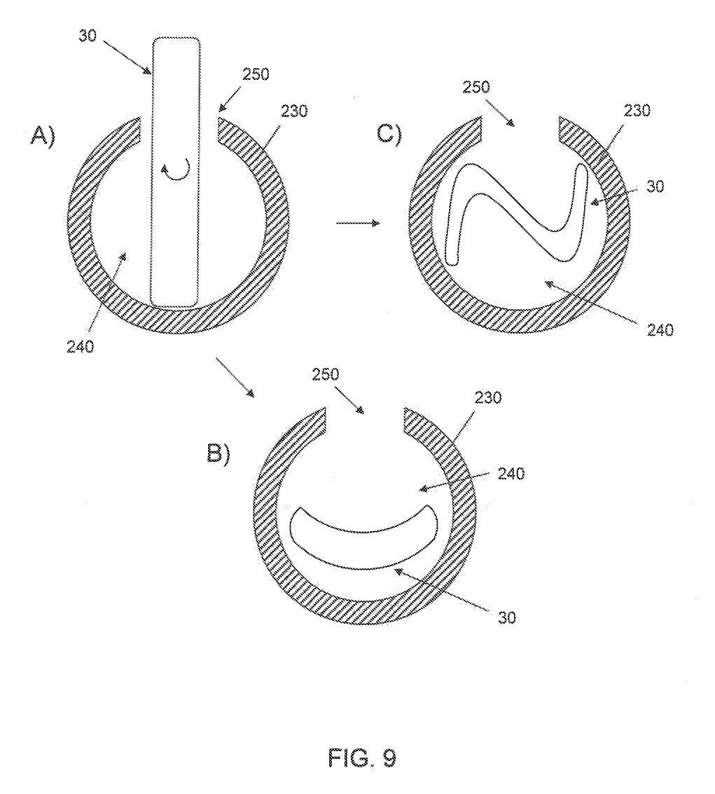

With reference now to FIGS. 7-9, an introducer 200 may include a body member 230 forming a longitudinal slit 250 extending from the proximal end 210 of the introducer towards the distal end 220 to facilitate entry of the paddle 30 into the lumen 240 of the introducer 200. The longitudinal slit 250 preferably has a length greater than the length of the paddle shaped portion of the lead. In some embodiments, the longitudinal slit 250 extends the length of the introducer 200 to the distal end 220 (see FIG. 8). As shown in FIG. 9, the lead may be axially rotated relative to the introducer to cause the paddle portion 30 of the lead to be fully received by the lumen 240 of the introducer. As the lead is rotated relative to the introducer, a sidewall of the body member 230 engages a surface of the paddle 30 causing the paddle 30 to bend as it enters the lumen 240 (see FIG. 9B). As shown in FIG. 9C, depending on the flexibility of the paddle portion 30 of the lead, a portion of the paddle portion 30 may frictionally engage the interior surface of the body 230 of the introducer and fold under itself as the lead is rotated relative to the introducer. Regardless of whether the paddle portion 30 of the lead bends as depicted in FIG. 9B or FIG. 9C or otherwise, the relative rotation of the lead caused the paddle portion 30 to be fully received in the lumen 240 of the introducer. Edges of the paddle 30 that are forced against the introducer may be rounded to a suitable extent to reduce the risk of cutting or damaging the paddle as is it inserted in or advanced through the introducer.

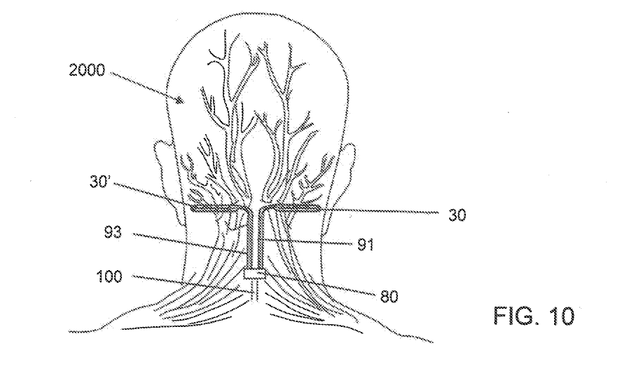

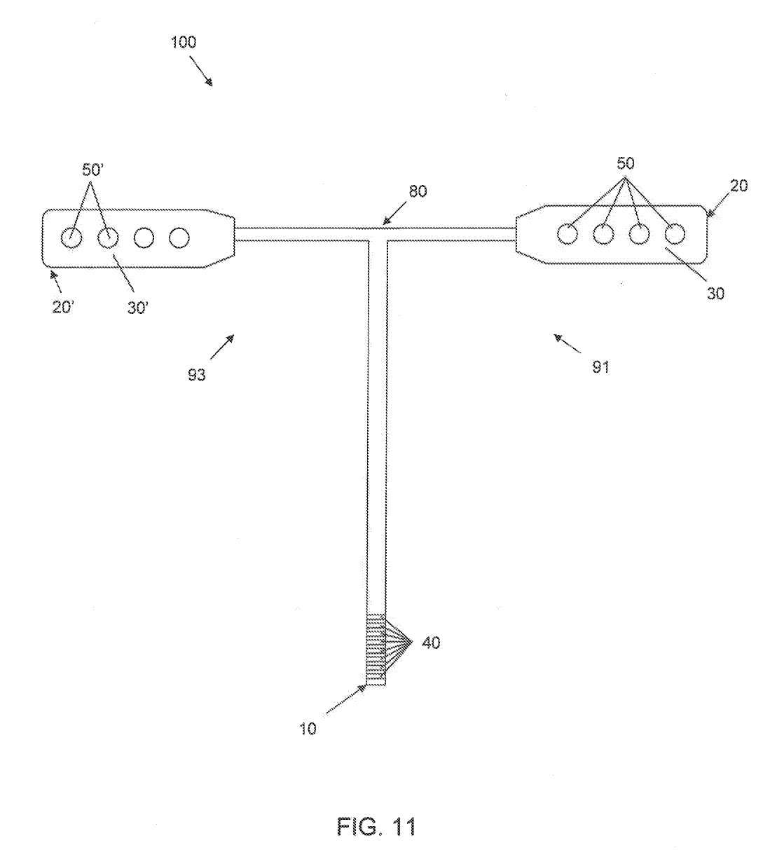

Referring now to FIG. 10, a bifurcated lead 100 may be employed to bilaterally apply electrical stimulation signals to left and right occipital nerves 2000 of a patient. The bifurcated lead 100 may be a lead as shown in FIG. 11. The bifurcated lead 100 has a branch point 80 between the proximal 10 and distal 20 ends. The lead 100 further includes first 91 and second 93 branches that extend from the branch point 80 to first 20 and second 20′ distal ends. Paddle shaped portions 30, 30′ including electrodes 50, 50′ form the distal portion of the first 91 and second 93 branches. The branches 91, 93 proximal the paddle-shaped portions 30, 30′ have outer diameters that are substantially uniform. The lead 100 depicted in FIG. 11 includes a number of contacts 40 equal to the cumulative number of electrodes 50, 50′ in both paddle portions 30, 30′.

Introduction of such bifurcated leads 100 generally cannot be accomplished with the use of standard introducers because the introducer cannot be withdrawn over the lead, as the introducer will get hung up at the branch point. Accordingly, introducers having longitudinal slits extending their length; e.g. as depicted and described with regard to FIG. 8, may be employed to deliver bifurcated paddle leads. The width of the slit of the introducer should be greater than the outer diameter of the lead distal the branch point 80 and proximal the paddle portion 30, 30′ to allow the introducer to be removed over the lead at or distal the branch point 80.

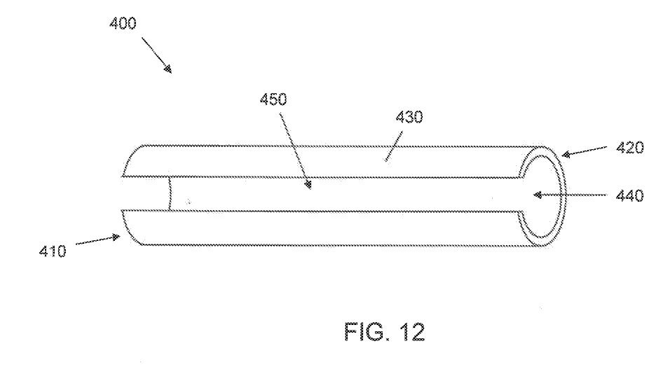

In many cases the branches 91, 93 of bifurcated leads cannot be rotated independently to cause paddle portions 30, 30′ to enter lumens of introducers. Even if such rotation was possible, it may be undesirable in many situations as twisting of a lead without free movement of the proximal end 10 can cause twisting of the lead body. In the case of implantation of bifurcated paddle leads through an introducer, it may be desirable to use a tool as depicted in FIG. 12. The tool 400 has a body member 430 that includes a proximal end 410, distal end 420, and defines a lumen 440 extending from the proximal end 410 towards the distal end 420. In the depicted embodiment, the lumen 440 extends the length of the tool 400 to the distal end 420. Of course the lumen 440 need not extend the entire length of the tool 400 (e.g., as described above with regard to the introducer depicted in FIG. 7).

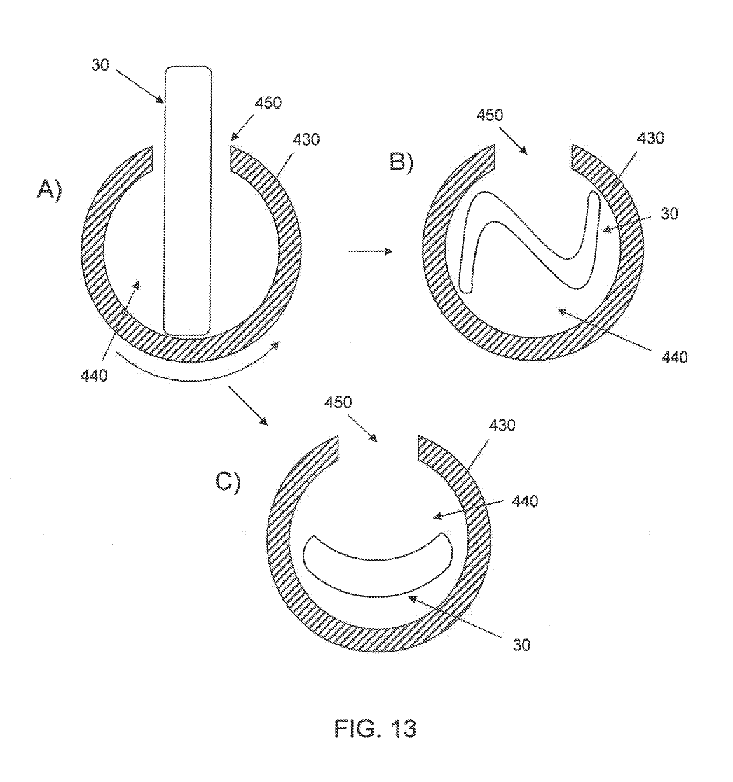

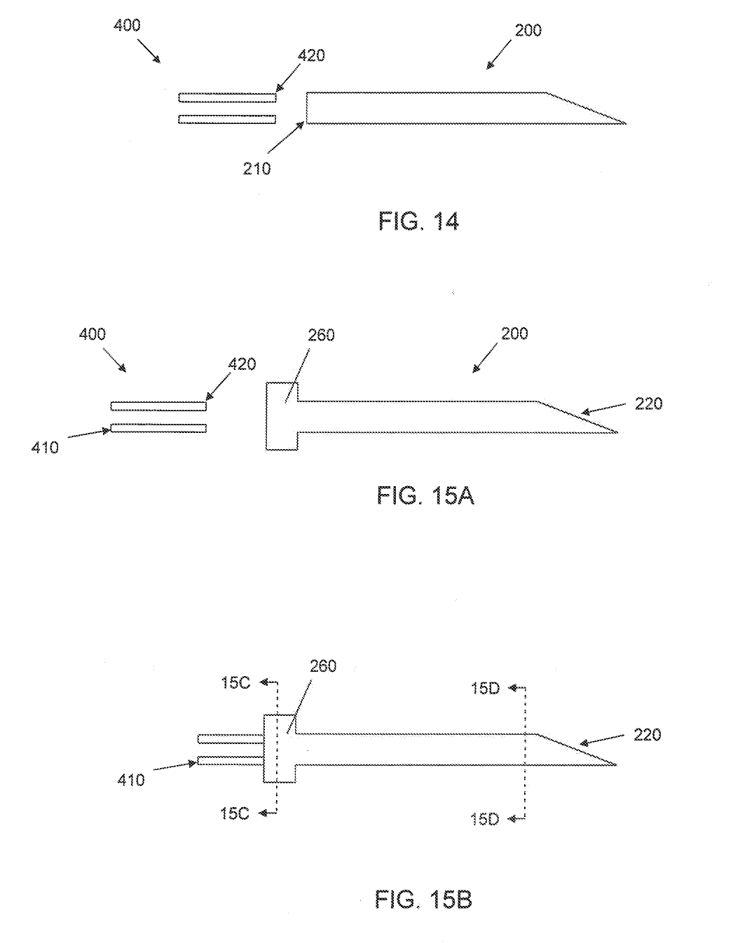

Referring now to FIG. 13, a portion of the paddle portion 30 of a bifurcated or non-bifurcated lead is inserted into the lumen 440 of the tool via the longitudinal slit 450 formed by the body member 430 (FIG. 13A). The tool 400 may be axially rotated relative to the paddle portion 30 such that a side wall of the body member 430 forming the slit 440 engages a surface of the paddle portion 30 to cause the paddle 30 to bend and be fully received by the lumen 440 (e.g., as shown in FIG. 14B or FIG. 14C). Once the paddle portion 30 of the lead is fully received by the lumen 440 of the tool, the lumen 440 of the tool may be axially aligned with the lumen of an introducer and the lead may be advanced through the lumen of the tool into the lumen of the introducer.

For example and referring to FIG. 14, the distal end 420 of the tool 400 may be placed in contact with or in proximity to the proximal end 210 of an introducer 200. The lumens of the introducer 200 and tool 400 should be generally aligned. A lead received in the lumen of the tool 400 may then be advanced through distally through the lumen of the tool 400 and into the lumen of the introducer 200.

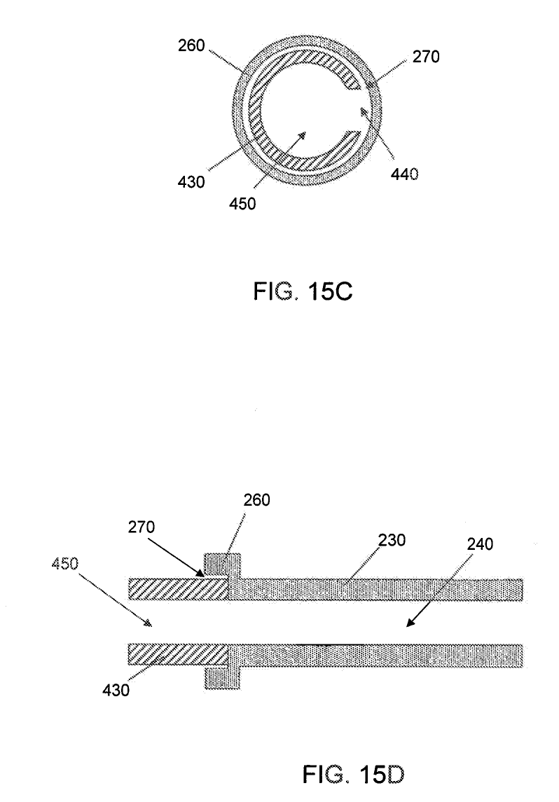

With reference to FIGS. 15A-D, an introducer 200, whether or not having a longitudinal slit, may include a hub 260 at its proximal end. The hub 260 forms a hub lumen 270 that is axially aligned with, and in communication with, the lumen 240 of the introducer. The hub lumen 270 is configured to receive the distal end 420 of the tool 400 and facilitates aligning the tool lumen 440 with the introducer lumen 240. The inner diameter of the hub 260 defined by the hub lumen 270 is the same as or larger than the outer diameter of the distal end of the tool 400. In various embodiments, the outer diameter of the tool 400 is substantially uniform over its length. The inner diameter of the tool 400 defined by the tool lumen 440 is substantially the same as the inner diameter of the introducer 200 defined by the introducer lumen 240. As shown in the embodiment depicted in FIG. 15D, a shoulder may be formed in a region of transition from the hub 260 to the distal portion of the introducer. The distal end of the tool 400 may abut the hub to further facilitate alignment of the tool with the introducer. While the proximal end 410 of the tool 400 is shown extending from the hub 260 when the tool 400 is inserted in the hub 260 (see FIG. 15B and 15D), the hub 460 may have a length longer than the length of the tool 400.

In various embodiments, the hub of the introducer 200 is configured to be operably coupled to an external electrical signal generator (not shown) so that proper positioning of the distal end 220 of the introducer 200 can be verified.

A tool 400 as described herein may be made of any suitable material, such as a metallic material or a polymeric material. Suitable metallic materials include stainless steel, titanium alloys, or the like. Suitable polymeric materials include polysulfone, polycarbonate, high density polyethylene, or the like. Tools 400 may be molded or otherwise formed. Longitudinal slits may be formed in the tools during initial manufacturing processes such as molding during subsequent processing steps, e.g. etching, cutting or stamping.

Introducers as described herein may be made in accordance with standard manufacturing techniques or may be purchased from a commercial source and used as is or modified appropriately. In many embodiments, introducers are fonned from stainless steel.

While implantation of leads to apply electrical signals to occipital nerves has been described above, it will be understood that the devices, systems and methods described herein may be used to implant surgical or paddle leads in nearly any location for nearly any purpose. Thus, embodiments of SYSTEM AND METHOD FOR IMPLANTING A PADDLE LEAD are disclosed. One skilled in the art will appreciate that the leads, extensions, connectors, devices such as signal generators, systems and methods described herein can be practiced with embodiments other than those disclosed. The disclosed embodiments are presented for purposes of illustration and not limitation.

What is claimed is:

1. A system comprising:
a tool having a body member defining a proximal end, a distal end, a lumen extending from the proximal end to the distal end, and a longitudinal slit in communication with the lumen and extending from the proximal end towards the distal end, the longitudinal slit having
a length;
a lead having
(i) a proximal end including a contact for operably coupling the lead to an active electrical medical device, and
(ii) a paddle-shaped distal end portion including an electrode electrically coupled to the contact, the paddle-shaped portion having a length; and
an introducer having a body member defining a proximal end, a distal end, and a lumen extending from the proximal end to the distal end,
wherein the tool and lead are configured such that axial rotation of the tool relative to the lead when the paddle portion of the lead is received by the slit causes the paddle portion of be fully received by the lumen, and
wherein the tool, the lead and the introducer are configured such that the paddle portion of the lead is advanceable distally through the lumen of the tool and into the lumen of the introducer when the lumens of the tool and the introducer are axially aligned.

2. A system according to claim 1, wherein the length of longitudinal slit of the tool is greater than the length of the paddle-shaped portion of the lead.

3. A system according to claim 1, wherein the longitudinal slit of the tool extends from the proximal end to the distal end of the tool.

4. A system according to claim 1, wherein the introducer comprises a hub at the proximal end of the introducer, wherein the hub forms a hub lumen axially aligned with and in communication with the lumen of the introducer, and wherein the hub lumen is configured to receive the distal end of the tool and to axially align the tool lumen with the introducer lumen when the distal end of the tool is received by the lumen.

5. A system according to claim 4, wherein the hub is configured to be operably coupled to an external active medical device.

6. A system according to claim 1, wherein the lead is bifurcated and includes a branch point between the proximal end and the distal end such that the lead
(i) has a first branch that includes the paddle-shaped portion and that extending from the branch point to the distal end, and
(ii) has a second branch extending from the branch point to a second distal end.

7. A system according to claim 6, wherein the introducer has a longitudinal slit extending from the proximal end to the distal end of the introducer.

8. A system according to claim 7, wherein the first branch proximal the paddle portion has an outer diameter and the longitudinal slit of the tool has a width greater than the outer diameter of the first branch proximal the paddle portion.

9. A system according to claim 1 further comprising an electrical signal generator configured to be operably coupled to the contact of the lead.

10. A method comprising:
inserting a paddle portion of a lead into a longitudinal slit of a tool having a lumen in communication with the slit;
axially rotating the tool relative to the paddle portion of the lead to cause the paddle portion of the lead to enter the lumen;
axially aligning the lumen of the tool with a lumen of an introducer; and
advancing the paddle portion of the lead through the lumen of the tool and into the lumen of the introducer.

11. A method according to claim 10, further comprising placing the introducer in a patient such that the introducer is positioned to deliver the paddle portion of the lead to the target tissue.

12. A method according to claim 10, further comprising withdrawing the introducer over the lead.

13. A method according to claim 10, wherein axially aligning the lumen of the tool with the lumen of the introducer comprises introducing a distal portion of the tool into a proximal hub of the introducer.

14. A method for applying an electrical signal to an occipital nerve of a patient, comprising:
positioning a distal end of an introducer in proximity to an occipital nerve of a patient, the introducer having a proximal end, the distal end, and a lumen extending from the proximal end to the distal end;
inserting a paddle portion of a lead into a longitudinal slit of a tool having a lumen in communication with the slit;
axially rotating the tool relative to the paddle portion of the lead to cause lumen of the tool to fully receive the paddle portion of the lead;
axially aligning the lumen of the tool with a lumen of the introducer, advancing the paddle portion of the lead through the lumen of the tool and into the lumen of the introducer;
distally advancing the paddle portion of the lead through the lumen of the introducer until the paddle portion of the lead is in proximity to the distal end of the introducer and the occipital nerve;
withdrawing the introducer from the patient over the lead; and
operably coupling the lead to an electrical signal generator such that a signal generated by the signal generator is deliverable to the occipital nerve via the paddle portion of the lead.

15. A method for implanting a lead having a distal paddle-shaped portion via an introducer, comprising:
bending the distal paddle-shaped portion of the lead;
inserting the bent distal paddle-shaped portion of the lead into a lumen of an introducer.

16. A method comprising:
inserting a paddle portion of a lead into a longitudinal slit of an introducer,
axially rotating the introducer to cause the paddle portion of the lead to enter a lumen of the introducer, the lumen being in communication with the longitudinal slit.
